(19) United States
(12) Patent Application Publication
(10) Pub. No.: US 2023/0255809 A1
(43) Pub. Date: Aug. 17, 2023

(54) DEVICES, SYSTEMS, AND METHODS FOR ENDOSCOPIC POSITIONING

(71) Applicant: BOSTON SCIENTIFIC SCIMED, INC., Maple Grove, MN (US)

(72) Inventors: Gonzalo Jose Saenz Villalobos, Alajuela (CR); Gian Franco Loo Fuchs, San Jose (CR)

(73) Assignee: BOSTON SCIENTIFIC SCIMED, INC., Maple Grove, MN (US)

(21) Appl. No.: 18/108,169

(22) Filed: Feb. 10, 2023

Related U.S. Application Data
(60) Provisional application No. 63/309,236, filed on Feb. 11, 2022.

(51) Int. Cl. A61F 2/958; A61B 17/11

(52) U.S. Cl. CPC A61F 2/958; A61B 17/11; A61B 2017/1125

(57) ABSTRACT

A locator system deployable at a target site in a first anatomical structure and identifiable from within a second anatomical structure. The locator system includes a signal generator and a tissue-engagement member. The tissue-engagement member engages tissue at the target site so that a delivery system may deliver the locator system and be withdrawn without disturbing the position of the locator system with the tissue-engagement member engaging tissue at the target site. The locator system may be deployed at a target site within a patient's intestines, and identified from within the patient's stomach to create an anastomosis between the stomach and the target site.

### CROSS-REFERENCE TO RELATED APPLICATIONS

This application claims the benefit of priority of U.S. Provisional Application No. 63/309,236, filed Feb. 11, 2022, the entire disclosure of which is hereby incorporated by reference herein for all purposes.

### FIELD

The present disclosure relates generally to the field of medical devices for use within a body. In particular, the present disclosure relates to medical devices, systems, and methods for use with endoscopic procedures. Even more particularly, the present disclosure relates to medical devices, systems, and methods for facilitating positioning of a device within the body, such as facilitating identification of the location of a target site within the body.

### BACKGROUND

Viewing, locating, and manipulating anatomies, devices, and/or anatomies containing one or more devices from within a body may be difficult. During a procedure within the body, such as an endoscopic procedure within a body lumen (not involving cutting open the body), a medical professional may need to externally locate a particular anatomical structure of and/or position within the body. Locating a desired anatomical structure of and/or position within a body may be difficult due to a lack of or low amount of illumination, and/or intervening anatomy, and/or the shape and/or configuration of various portions of the body. For instance, procedures within body lumens such as the intestines present lengthy regions of the anatomy which may be difficult to differentiate from outside the body. Various locating devices may be provided within the body. However, manipulation of devices within the body with respect to such locating devices (e.g., to perform a procedure with the device being manipulated, and/or to withdraw a delivery device for the locating device) may cause such locating devices to be displaced, thereby interfering with the purpose and function of such locating devices.

It is with respect to these and other considerations that the present improvements may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a locator system includes a flexible elongate member, a beacon mounted on the flexible elongate member, and a tissue engagement member configured to engage tissue at a target site to anchor the locator system with respect to the target site.

In some embodiments, the tissue engagement member is expandable to engage tissue at the target site to anchor the locator system with respect to the target site. In some embodiments, the tissue engagement member is an inflatable balloon. In some embodiments, the tissue engagement member is an expandable stent. In some embodiments, the tissue engagement member is mounted on the flexible elongate member.

In some embodiments, the tissue engagement member has a pair of grasper arms configured to grasp tissue at the target site therebetween.

In some embodiments, the locator system further includes a controller extending along the flexible elongate member and actuatable to shift the tissue engagement member between a delivery configuration and a tissue-engaging configuration. In some embodiments, the tissue engagement member is inflatable, and the controller is an inflation lumen through the flexible elongate member. In some embodiments, the tissue engagement member is inflatable, and the controller is an inflation lumen within an inflation line extending along the flexible elongate member. In some embodiments, the controller is a sheath extending about the flexible elongate member; and the tissue engagement member is an expandable stent positioned within the controller in the delivery configuration and expandable into the tissue-engaging configuration when outside the controller. In some embodiments, the tissue engagement member has a pair of grasper arms configured to grasp tissue at the target site therebetween.

In accordance with various principles of the present disclosure, a locator system includes a beacon, a tissue engagement member, and a controller actuatable to shift the tissue engagement member between a delivery configuration in which the tissue engagement member is compact and deliverable transluminally to a target site, and a tissue-engaging configuration in which the tissue engagement member engages tissue at the target site to anchor the beacon with respect to the target site.

In some embodiments, the locator system includes a flexible elongate member on which the beacon and the tissue engagement member are mounted.

In some embodiments, the tissue engagement member is an inflatable balloon, and the controller is an inflation lumen in fluid communication with the balloon.

In some embodiments, the controller is a sheath; and the tissue engagement member is an expandable stent shiftable between a delivery configuration when within the sheath and an expanded tissue-engaging configuration when outside the sheath.

In some embodiments, the tissue engagement member includes a pair of jaws movable by the controller between a closed configuration in which the jaws are in a delivery configuration or grasping tissue therebetween, and an open configuration in which the jaws may engage tissue therebetween.

In accordance with various principles of the present disclosure, a method of locating a target site in a first anatomical structure from within a second anatomical structure includes delivering a locator system to the target site with a delivery system, anchoring the locator system with respect to the target site by engaging a tissue engagement member of the locator system with tissue at the target site, withdrawing the delivery system from the locator system to leave the locator system in place anchored with respect to the target site, and locating and identifying the locator system from the second anatomical structure, through a wall of at least the second anatomical structure.

In some embodiments, the method includes forming an anastomosis between the first anatomical structure and the second anatomical structure along the target site.

In some embodiments, the method includes expanding the tissue engagement member from a compact delivery configuration to an expanded configuration to engage tissue at the target site to anchor the locator system with respect to the target site.

In some embodiments, the method includes anchoring the locator system to the target site by grasping tissue at the target site between grasper arms of the tissue engagement member.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an implantable locator system formed in accordance with various aspects of the present disclosure and positioned in a schematic representation of a gastrointestinal environment.

FIG. 2 illustrates a view similar to that of FIG. 1, but with the large intestine shown in phantom, and with an example of an embodiment of a locator system deployed at a target site, and a delivery system delivering instruments to reach the target site.

FIG. 3 illustrates a view similar to that of FIG. 2, with instruments illustrated being delivered in FIG. 2 reaching the target site.

FIG. 4 illustrates an isolated view of the portions of the stomach and jejunum illustrated in FIG. 3 as being drawn together, with an anastomosis therebetween.

FIG. 5 illustrates a view similar to that of FIG. 4, but with a tissue approximator holding the stomach and jejunum tissue walls in apposition.

FIG. 6 illustrates a perspective view of an example of an embodiment of a locator system formed in accordance with various principles of the present disclosure.

FIG. 7 illustrates a perspective view of an example of an embodiment of a locator system formed in accordance with various principles of the present disclosure, being delivered to a target site.

FIG. 8 illustrates a perspective view of an example of an embodiment of a locator system as in FIG. 7 in a deployed position.

FIG. 9 illustrates a perspective view of an example of an embodiment of a locator system formed in accordance with various principles of the present disclosure, being delivered to a target site.

FIG. 10 illustrates a perspective view of an example of an embodiment of a locator system, such as in FIG. 9, in a deployed position.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore.

A number of medical procedures require identification of the location of an anatomical structure, such as an organ or a tissue wall, and then delivering medical instruments to such location to perform a procedure on or at the identified location (e.g., “target site” or “target tissue” or “target tissue site”, etc., the present disclosure being applicable to any such location, reference to any one such designation being applicable to the other designations without intent to limit unless otherwise indicated). It may desirable to be able to locate the identified location at a later time (e.g., after the delivery system for the locator system has been removed), and/or from another location within the body, such as to perform a procedure after identification of the anatomical location. Non-limiting examples of procedures which may be performed include various medical procedures which involve moving a tissue wall (e.g., a body lumen wall or the wall of an organ) to a desired position, such as relative to another tissue wall (e.g., a body lumen wall or the wall of an organ). For instance, various procedures may be performed by entering the gastrointestinal (GI) tract through a first organ or structure (such as the esophagus, stomach, duodenum, small intestine, large intestine, or peritoneal cavity), and delivering an anchor or stent to adjacent organs or lumen or tissue structures (such as an adjacent portion of the GI tract, the bile duct, the pancreatic duct, the gallbladder, the pancreas, cysts, pseudocysts, abscesses, and the like). Typically, it is necessary to penetrate both an access tissue wall (e.g., a wall of an organ or a first body lumen), through which access is established, and a second tissue wall (e.g., of a wall of an organ or a second body lumen) along or adjacent or at the target for the procedure. A stent or other tissue anchor may be deployed between adjacent body lumens, organs, or other structures, such as to maintain tissue walls in apposition, and/or to create an anastomosis, as indicated by the procedure. Tissue anchors may be used, in addition to the stent(s), to secure adjacent tissues or organs, such as before a stent is deployed, and may be left in place after the stent has been deployed.

Endoscopic procedures have been used to create a connection, such as an anastomosis, between the stomach and a certain part of the intestines through a gastroenteral anastomosis. For example, conditions of a disease may be benefitted by bypassing a portion of the duodenum, such as by creating an anastomosis, e.g., about 150 cm or greater from the pylorus, between the stomach and the small intestines (with a distal portion of the duodenum, or with the jejunum). In gastric outlet obstruction, a gastrojejunostomy serves the purpose of draining the contents of the stomach into the jejunum below/distal to the obstructed/dysfunctional duodenum. A gastrojejunostomy procedure may also serve as a minimally invasive and possibly reversible treatment option for patients with metabolic disease, by creating an anastomosis between the stomach and the jejunum to bypass the duodenum, with accompanying desired metabolic effects. In this manner, absorption of stomach contents (e.g., food and other nutrients) in the duodenal portion may be bypassed and nutrients from such contents may not be absorbed, or uptake or absorption may delayed, as such contents travel from the stomach through the small bowel, promoting patient weight loss and possible controlling or resolving type-2 diabetes.

Gastroenteral anastomoses, and other procedures within the body, may be created surgically, either endoscopically (gastroscopically or laparoscopically) or through an open surgical procedure. Endoscopic procedures, such as gastroenteral anastomoses, present various challenges, including the need to endoscopically locate a desired position, such as in the intestines via the gastric lumen. Such position or location may be referenced herein as a “target site” or “target tissue site”. Ultrasound and/or fluoroscopy procedures provide images through anatomical walls (e.g., the gastric and enteral walls). However, although ultrasound is useful for imaging tissue, ultrasound may not image inorganic materials used to identify tissue as readily. Although fluoroscopy is well suited for viewing dense materials, such as those from which medical instruments are made, contrast mediums used with fluoroscopy may dissipate as the target tissue is located and may thus need to be reintroduced. It will be appreciated that terms such as instruments, tools, devices, etc., may be used interchangeably herein without intent to limit.

Another solution involves several devices that work in the following manner. First, a beacon system, which includes a light at the distal end of a flexible elongate member (such as a guidewire or tubular element), is inserted, such as with an endoscope, to the desired location for the procedure (e.g., where the connection of the intestines with the stomach is intended to be made). The endoscope is withdrawn, leaving the light in place. Then, the endoscope is re-inserted into the body (e.g., parallel to the flexible elongate member with the light at the distal end thereof) and one or more instruments for performing the procedure are inserted, such as to make an incision is made in the stomach wall and into the peritoneum. The light is visualized, or otherwise located, to identify the target site for the procedure and the procedure may thus be performed at the desired target site. For instance, once in the peritoneum, the physician locates the desired section of the small intestines with the aid of the light therein that can be seen from outside the intestinal wall. The outside of the small intestines is grasped and brought together with the stomach. A stent may then be deployed to connect the small intestines and the stomach, thereby creating the anastomosis.

The above-described solution using a light to locate a target site for a procedure also presents some challenges. For instance, during the initial steps of the procedure, after the guidewire is located at the desired location, the endoscope needs to be retracted from the body while maintaining the location of the light within the body at the desired location. Retraction of the endoscope must be coordinated with advancement of the flexible elongate member on which the light is positioned in order to maintain the original, desired position of the light. Such procedure may be difficult to perform accurately, and may be prone to affect the final position of the light, and thus the location at which the one or more instruments for performing the procedure are to be advanced (e.g., portion of the small intestines at which the anastomosis with the stomach is to be formed), which is an important factor for the success of the process.

The present disclosure relates to devices, systems, and methods useful in performing a procedure, e.g., an endoscopic, laparoscopic, and/or open surgical procedure, within the body by initially identifying the target site for the procedure with a locator system, deploying the locator system at the target site, and then locating the locator system to perform the procedure at the desired target site identified by the locator system. In some aspects, the devices, systems, and methods may be used to create an anastomosis such as a gastrojejunal anastomosis. For example, devices and systems described herein may aid gastrojejunal anastomosis placement by reliably and repeatably locating a desired position in a patient's gastrointestinal system, e.g., distinguishing a position in the jejunum, such as proximal or distal or adjacent to the Ligament of Treitz. Additionally, devices and systems herein may allow for a medical professional to locate, grasp, hold, and/or cut a portion of the stomach and small bowel during a gastrojejunal anastomosis procedure. Optionally a stent or other conduit may be placed across the bypass bridging the walls of the stomach and jejunum where the openings are created. The stent or conduit may assist with establishing or maintaining the anastomosis open until it is stable. The stent may or may not be subsequently removed.

It will be appreciated that various principles of the present disclosure may be applied to devices, systems, and methods for performing other procedures within a patient's body with the use of a locator system and locator system delivery and deployment system as disclosed herein. Thus, although the systems and devices and methods described herein are described with respect to a gastrointestinal system, it may be understood that devices and systems and methods in accordance with the present disclosure may be advantageous for use in any other procedures, such as, without limitation, those involving grasping, manipulation, or cutting of tissue (e.g., a body lumen and/or other sensitive tissue structures). Moreover, it should be understood that the systems and devices and methods described herein may be used with other regions of the anatomy, such as anywhere selective location of tissue is through other tissue walls and/or is blind.

In accordance with various principles of the present disclosure, a locator system is delivered by a delivery system to a target site, and is affixed to target tissue at the target site (such as to be located from another region of the body). Such engagement of the locator system with tissue anchors the locator system with respect to the target site, thereby removing the need for coordinating removal of the delivery system with maintaining the locator device in the desired location at the target site. It will be appreciated that terms such as couple, engage, grasp, hold, clasp, clip, anchor, affix, maintain, secure, etc. (and other grammatical forms thereof) may be used interchangeably herein without intent to limit. It will further be appreciated that reference may be made herein to locator, beacon, guide, signal, emitter, light, etc., interchangeably and without intent to limit.

In some embodiments, the locator system includes a tissue-engagement member configured to anchor the locator system with tissue at the target site. It will be appreciated that the tissue-engagement member may alternately be referenced herein as a tissue fastener or clip or other mechanical securing device (e.g., a hemostatic clip, clamp, grasper, basket, gripper, magnet, adhesive, etc.), without intent to limit. The tissue-engagement member may be shiftable between a delivery configuration (e.g., a compact configuration facilitating delivery of the locator system to the target site) and a tissue-engaging configuration (e.g., engaging tissue to anchor the locator system with respect to the engaged tissue). An actuator may be provided to shift the tissue-engagement member between the delivery configuration and the tissue-engaging configuration. It will further be appreciated that the tissue which is engaged by the tissue-engagement member at the target site may be at the location at which a procedure such as an anastomosis is to be performed, or distal or proximal to such procedure, depending on the procedure being performed and the technique being used and the medical professional's specifications.

Various embodiments of devices, systems, and methods for locating a position within a body will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers less than 1000 are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

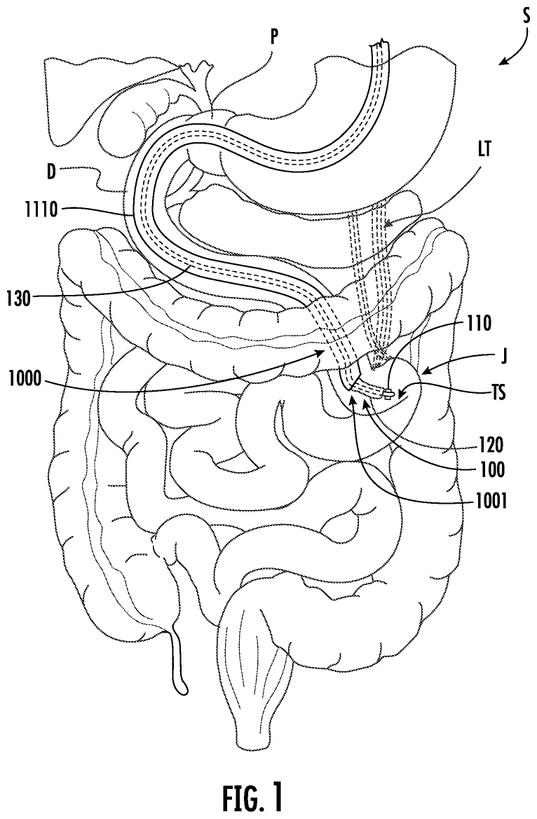

Turning now to the drawings, an example of an embodiment of a locator system 100 and delivery system 1000 configured to deliver the locator system 100 to a target site TS are illustrated in FIG. 1 in a schematic representation of a gastrointestinal system. It will be appreciated that principles of the present disclosure may be applied to other anatomical sites and structures, reference being made to gastrointestinal locations and structures for the sake of convenience and without intent to limit. It will further be appreciated that the tissue which is engaged by the tissue-engagement member at the target site may be at the location at which a procedure such as an anastomosis is to be performed, or distal or proximal to such procedure, depending on the procedure being performed and the technique being used and the medical professional's specifications. Moreover, it will be appreciated that reference to “at” is intended to include at and about the vicinity of e.g., along, adjacent, etc.). In the illustrated example of an environment, a locator delivery system 1000 may be inserted, such as through a natural orifice transluminal endoscopic surgery (NOTES) procedure (e.g., through the nose or mouth, and into the esophagus) into and through the stomach S, through the pylorus P, and into the duodenum D. In the example of an embodiment illustrated in FIG. 1, the locator delivery system 1000 includes a flexible elongate delivery member 1110 capable of being delivered to the target site TS through the body. The flexible elongate delivery member 1110 may be delivered through a lumen within a flexible elongate tubular member (e.g., a shaft, catheter, endoscope, etc.) known to those of ordinary skill in the art for transluminal delivery of devices through the body (in contrast with via open-surgery techniques). In some embodiments, the flexible elongate delivery member 1110 is delivered through an endoscope (not shown, but which may be any of a variety of endoscopes known to those of ordinary skill in the art) with visualization and/or imaging abilities to facilitate location of the target site TS.

The locator system 100 is advanced by the locator delivery system 1000 to the desired target site TS for the locator system 100. The locator delivery system 1000 and locator system 100 may be advanced with the use of a control handle at a proximal end of the locator delivery system 1000 (not shown, but which may be any suitable control handle known to those of ordinary skill in the art, the details of which do not limit and are not critical to the present disclosure). Peristaltic migration through the GI system may also assist with advancing the locator delivery system 1000 and locator system 100. In the example of an environment illustrated in FIG. 1, the target site TS is in the jejunum J, such as adjacent, proximal, or distal to the Ligament of Treitz LT. A section of the jejunum J may be selected as a “target site TS” at a distance from the pylorus P at which an anastomosis between the jejunum J and the stomach S is to be formed (such as determined by a medical professional). The locator delivery system 1000 may assist in identifying and selecting the target site TS, such as with the use of optical/visualization elements known to those of ordinary skill in the art (e.g., a camera, scope, fiber optics, fluoroscopy, etc., the details of which do not limit and are not critical to the present disclosure). The locator system 100 is advanced by the locator delivery system 1000 to the target site TS. A beacon 110 on the locator system 100 serves to identify the target site TS so that the target site TS may be located by instruments in another anatomical position, such as within the stomach S, as described in further detail below. The beacon 110 may be any of a variety of devices emitting a signal locatable through anatomical tissue. In some embodiments, the beacon 110 includes one or more light-emitting-diodes (LED's).

In accordance with various principles of the present disclosure, once the locator system 100 has reached the target site TS, the locator system 100 is deployed by engaging a tissue-engagement member 120 of the locator system 100 (such as described in further detail below) with tissue at the target site TS. As such, the locator system 100 is anchored with respect to the target site TS, and the locator delivery system 1000 may be withdrawn from the locator system 100 without affecting the position of the locator system 100. Instruments for creating an anastomosis between the target site TS and a section of the stomach S (preferably in proximity to the target site TS in the jejunum J) may then be inserted into the body to create an anastomosis between the target site TS and the stomach S.

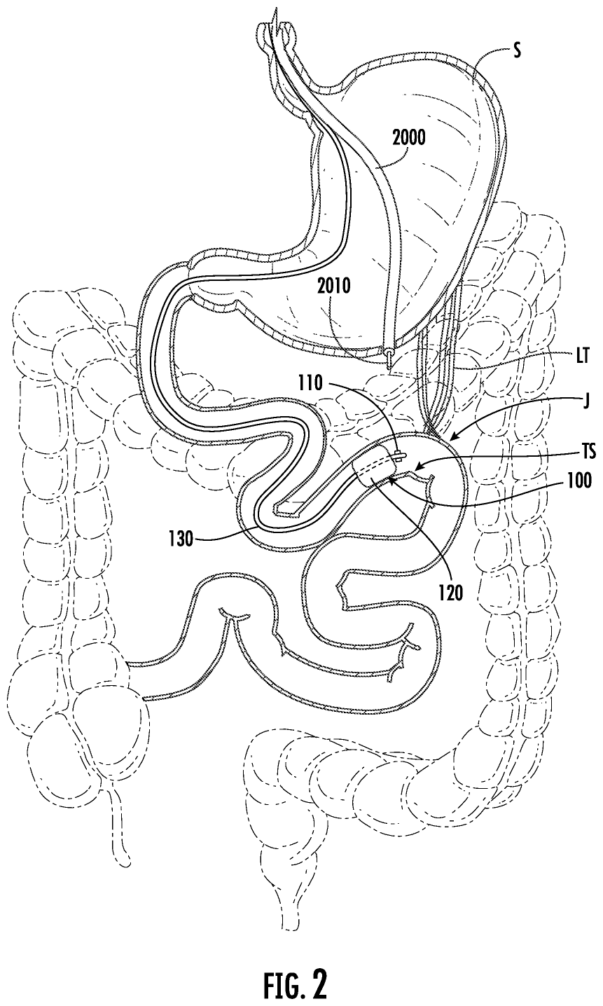

To create an anastomosis between the jejunum J and the stomach S, a delivery system 2000, such as illustrated in FIG. 2, may be inserted into the GI system, such as through a NOTES procedure, and into the stomach S. It will be appreciated that the delivery system 2000 used to create the anastomosis may include common delivery components as in the locator delivery system 1000. For instance, the endoscope may be used in the delivery system 2000 as in the locator delivery system 1000. The delivery system 2000 includes a visualization system by which the beacon 110 of the locator system 100 may be visualized to locate the target site TS. For instance, the delivery system 2000 may include an endoscope with a camera or other visualization system as known or heretofore known by those of ordinary skill in the art, the details of which do not limit and are not critical to principles of the present disclosure. Once the target site TS has been located, various instruments, tools, devices, etc. (such terms being used interchangeably herein without intent to limit) may be delivered by the delivery system 2000 to create an anastomosis between the target site TS and an appropriate location along the stomach S.

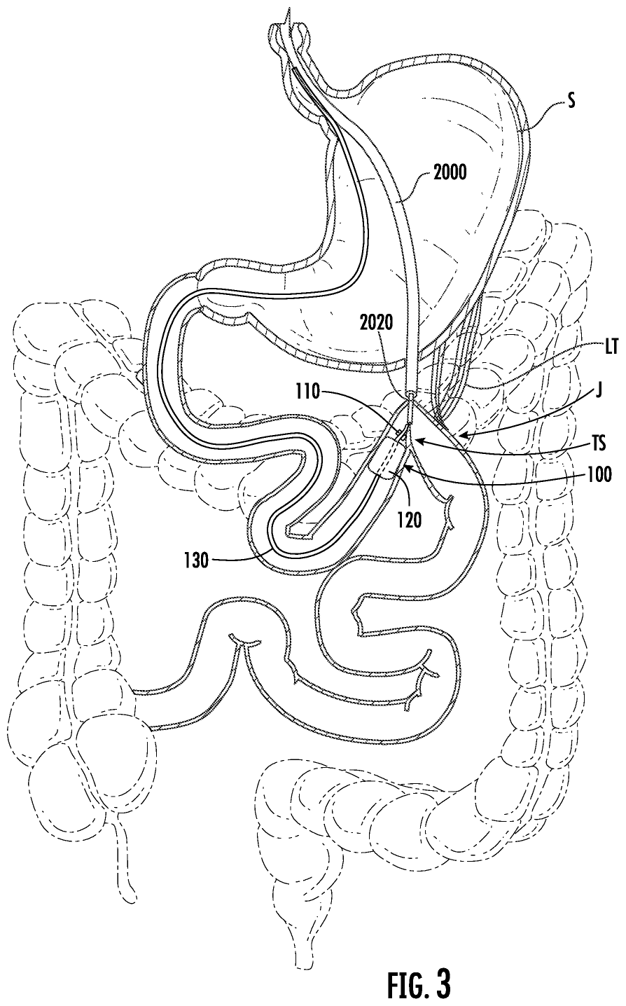

For instance, a cutting tool 2010 may be delivered by the delivery system 2000 to create an opening through the wall of the stomach S in the region of the target site TS (as guided by the beacon 110), as illustrated in FIG. 2. It will be appreciated that the term “cutting” is to be understood herein in the broad sense of creating an opening, and a “cutting tool” is to be understood as any tool known to those of ordinary skill in the art capable of creating an opening in tissue, such as a blade, cauterization tool (e.g., with a cauterization blade), needle, scissors, ablation device, other energy delivery device, etc., the present disclosure not being limited specifically to cutting in any narrow sense of such term. A grasping tool 2020 (any grasper known to those of ordinary skill in the art capable of grasping tissue, such as an end effector, clips, snare, etc.) may be delivered by the delivery system 2000 (e.g., through a working channel through which the cutting tool 2010 has been delivered, and after withdrawal of the cutting tool 2010; or through a different working channel of the delivery system 2000; or as a part of the cutting tool 2010; or otherwise), and extended through the hole in the stomach S cut by the cutting tool 2010, as illustrated in FIG. 3. The grasping tool 2020 is extended towards the jejunum J to grasp the portion of the jejunum J with the target site TS therein (as identified by the locator system 100), and to draw such portion of the jejunum J to the stomach S. The jejunum J may be held in position with respect to the stomach S and an anastomosis may be formed therebetween in any desired manner known or heretofore known by those of ordinary skill in the art.

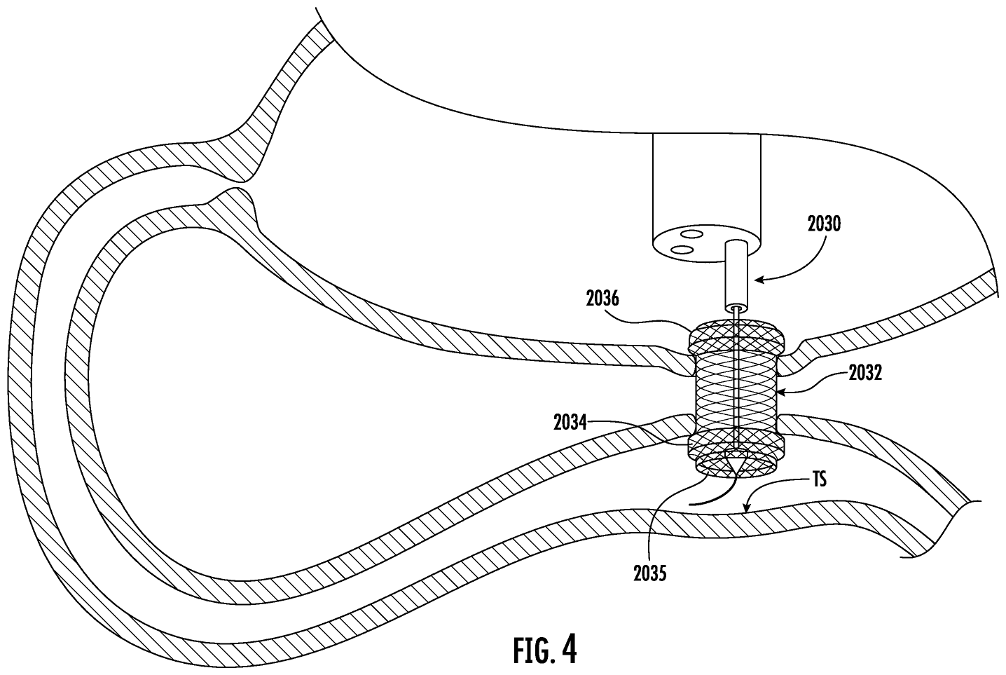

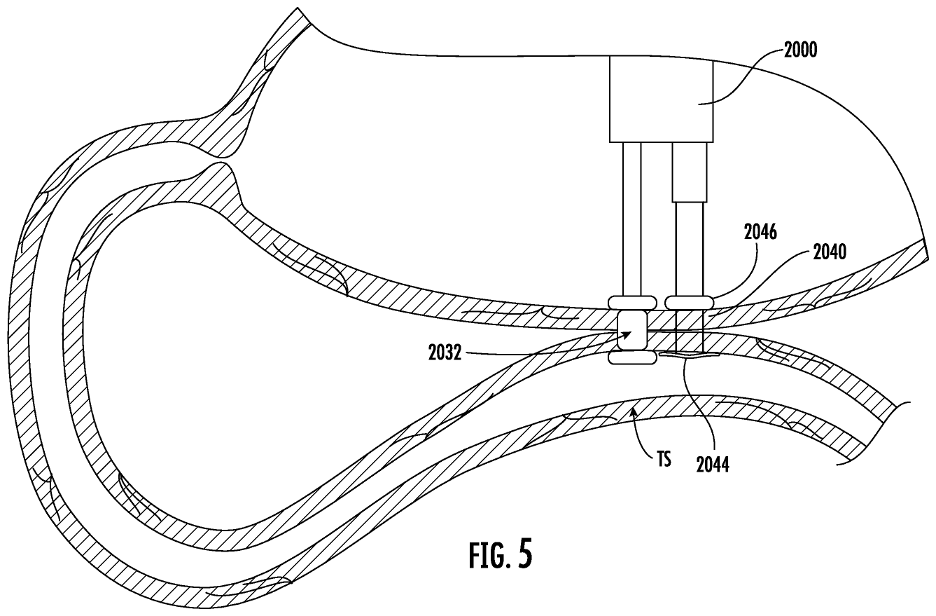

For instance, in some embodiments, an anastomosis system 2030 may be delivered to the apposed portions of the jejunum J and stomach S to form an anastomosis therebetween. In some embodiments, such as illustrated in FIG. 4, the anastomosis system 2030 may deliver a stent 2032 configured to hold the apposed portions of the jejunum J and stomach S together (e.g., with a retention member 2034 within the jejunum J, and a retention member 2036 within the stomach S) and to create an anastomosis therebetween (e.g., via a lumen 2035 through the stent 2032). In some embodiments, such as illustrated in FIG. 5, the jejunum J and stomach S may be held in close apposition by a tissue approximator 2040 for deployment of a stent 2032 through such tissue walls to form an anastomosis (such as described above with respect to the example of an embodiment illustrated in FIG. 4). In some embodiments, the tissue approximator 2040 extends through apposed tissue walls and is configured to hold tissue walls in apposition such as with an expanded tissue anchoring end 2044 within the jejunum J, and an expanded tissue anchoring end 2046 within the stomach S. A stent 2032 (such as described above with respect to the example of an embodiment illustrated in FIG. 4) may be deployed adjacent the tissue approximator 2040 to form the anastomosis as in the example of an embodiment illustrated in FIG. 5. The example of an embodiment of a tissue approximator 2040 as illustrated in FIG. 4 may be delivered by the delivery system 2000 and/or the anastomosis system 2030 (which may share features and/or structures). In some embodiments, the tissue approximator 2040 is deployed through the opening formed by the cutting tool 2010. In such instance, the anastomosis system 2030 may include a cutting tool to create an opening in apposed walls of the jejunum J and stomach S adjacent the tissue approximator 2040 as well as a deployment device to deploy the stent 2032 therethrough. It will be appreciated that once the anastomosis between the jejunum J and the stomach S has been formed, the locator system 100 may be removed, and thus optionally is not present, such as illustrated in FIG. 4 and FIG. 5.

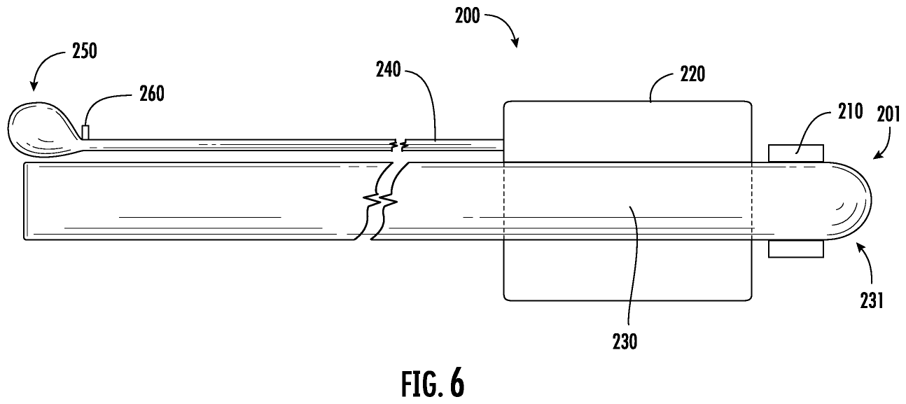

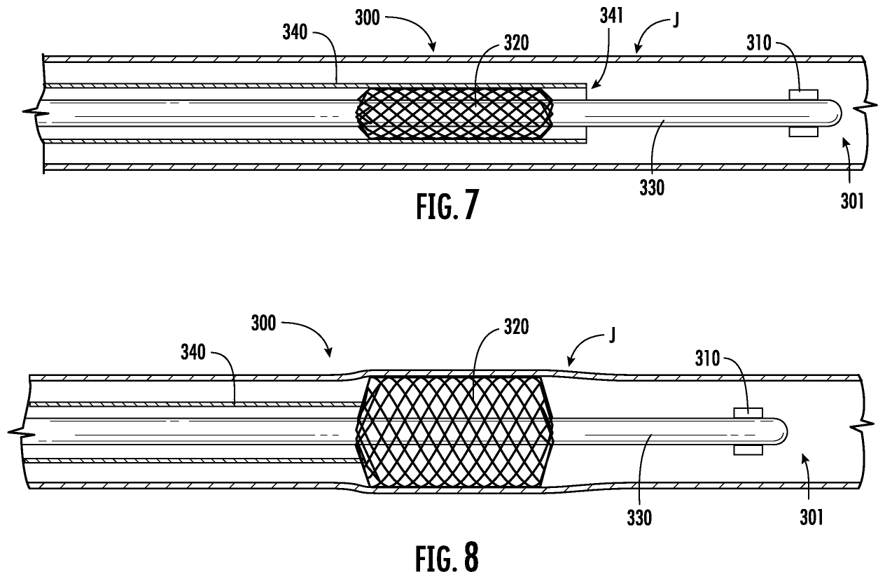

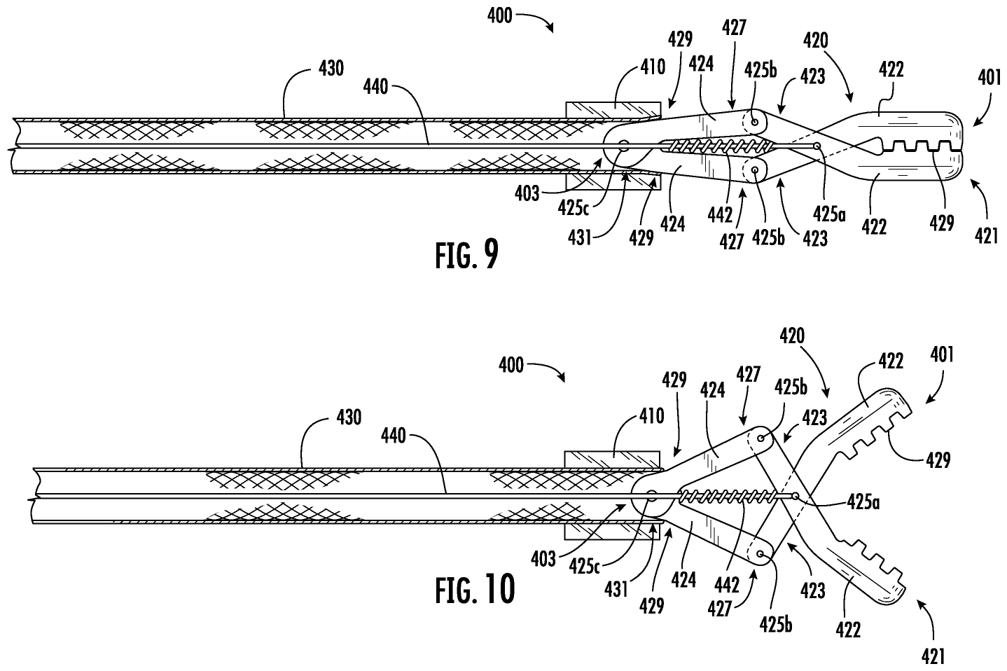

Examples of embodiments of locator systems 200, 300 formed in accordance with various principles of the present disclosure and used as described above are shown in further detail in FIG. 6, FIG. 7, FIG. 8, FIG. 9, and FIG. 10.

In the example of an embodiment of a locator system 200 illustrated in FIG. 6, the beacon 210 is adjacent the distal end 201 of the locator system 200, distal to the tissue-engagement member 220. The beacon 210 and tissue-engagement member 220 are illustrated as mounted on a flexible elongate member 230, such as along a distal end 231 thereof. Although the tissue-engagement member 220 is proximal to the beacon 210 (which mounted adjacent the distal end 231 of flexible elongate member 230), a reverse configuration is within the scope and spirit of the present disclosure as well. The flexible elongate member 230 may be a guidewire or other element known to those of ordinary skill in the art as capable of transluminal navigation through a body (e.g., through a GI system) to a target site TS, such as to deliver the beacon 210 to the target site TS (e.g., through a working channel or lumen within the delivery system 1000). The tissue-engagement member 220 is in the form of an expandable element. For example, in the example of an embodiment illustrated in FIG. 6, the tissue-engagement member 220 is an expandable balloon. However, other forms of expandable members capable of expanding to contact tissue wall adjacent a target site TS (e.g., to expand into contact with the interior of a portion of a jejunum J adjacent a target site TS) to anchor the locator system 200 with respect to the target site TS are within the scope and spirit of the present disclosure. Once the tissue-engagement member 220 has been expanded into contact with tissue walls (e.g., e.g., the interior of the jejunum J) at the target site TS, the delivery system 1000 may be withdrawn from the target site TS without disturbing the position in which the tissue-engagement member 220 anchors the beacon 210 for identification of the location thereof for performance of a further procedure.

A tissue-engagement member 220 in the form of an expandable balloon, as illustrated in FIG. 6, may be formed of a compliant or non-compliant biocompatible material, such as, without limitation, polymeric materials such as polyethylene terephthalate (PET), polyamides (such as nylon, polyamide homopolymers, polyamide copolymers, poly (ether-co-amide) copolymers), silicone, polyurethane, polyarylene sulfide, polyurethane block copolymers, block copolymer thermoplastic elastomers, polyether-block-amide copolymers, polyester-block-ether copolymers, including combinations and/or copolymers thereof. The tissue-engagement member 220 may be shifted between a delivery configuration in which tissue-engagement member 220 is in a compact configuration facilitating translumenal delivery, and an expanded tissue-engaging configuration. The locator system 200 includes an inflation lumen to control shifting of the tissue-engagement member 220 between the delivery configuration and the tissue-engaging configuration. In some embodiments, the flexible elongate member 230 has an inflation lumen extending therethrough and through which an inflation medium (e.g., a fluid such as air or saline solution) may be delivered to inflate a tissue-engagement member 220 in the form of an inflatable balloon. Alternatively, a separate inflation line 240, with an inflation lumen defined therethrough, may be provided, such as alongside the flexible elongate member 220. An air pump 250, to provide inflation medium to the tissue-engagement member 220, and a deflating valve 260, to allow release of inflation medium to deflate the tissue-engagement member 220 (such as to remove the tissue-engagement member 220 from the target site TS, such as upon completion of a procedure formed with respect to the target site TS), may be fluidly coupled with the tissue-engagement member 220, via the inflation line 240, as schematically illustrated in FIG. 6, or via an inflation lumen within the flexible elongate member 230.

Instead of an inflatable expandable member, as in the example of an embodiment illustrated in FIG. 6, a locator system 300 formed in accordance with various principles of the present disclosure may have an expandable member in the form of a stent, such as illustrated in FIG. 7 and FIG. 8. Like the example of an embodiment illustrated in FIG. 6, the illustrated example of an embodiment of a locator system 300 illustrated in FIG. 7 and FIG. 8 has a beacon 310 and an expandable tissue-engagement member 320 mounted on a flexible elongate member 330. Although the beacon 310 is distal to the tissue-engagement member 320, the reverse configuration is within the scope and spirit of the present disclosure. In addition, the locator system 300 includes a sheath 340 extending over at least the tissue-engagement member 320 in a delivery configuration, as illustrated in FIG. 7, such as to protect the tissue-engagement member 320 and/or to maintain the expandable tissue-engagement member 320 in a compact delivery configuration as it is delivered through the delivery system 1000 to the target site TS. In some embodiments, the beacon 310 may be delivered distal to the sheath 340. As illustrated in FIG. 8, once the beacon 310 has been delivered to the target site TS, the sheath 340 may be retracted proximally to allow the expandable tissue-engagement member 320 to expand into contact with tissue walls (e.g., the interior of the jejunum J) at the target site TS. As such, the delivery system 1000 may be withdrawn from the target site TS, in accordance with various principles of the present disclosure, without disturbing the position in which the tissue-engagement member 320 anchors the beacon 310 for identification of the location thereof for performance of a further procedure. The sheath 340 may be left in place to permit distal advancement over the tissue-engagement member 320 to return the tissue-engagement member 320 to a compact configuration for withdrawal and removal from the target site TS, such as upon completion of a procedure performed with respect to the target site TS.

The tissue-engagement member 320 may be a self-expanding device such as a stent as known or heretofore known to those of ordinary skill in the art For instance, the strut members may be formed of shape-memory or heat-formable material (e.g., Nitinol or Elgiloy®) so that the tissue-engagement member 320 returns to a pre-shaped expanded configuration from a collapsed configuration upon withdrawal of the sheath 340, which maintains the tissue-engagement member 320 in a compact delivery configuration. More particularly, the expandable tissue-engagement member 320 may be formed in a variety of manners, such as from one or more strut members combined to form a rigid and/or semi-rigid stent structure. The strut members may be formed of one or more wires or filaments which are braided, wrapped, intertwined, interwoven, weaved, knitted, looped (e.g., bobbinet-style), knotted, or the like to form an expandable and contractable scaffold configuration. The wires or filaments may be formed from a variety of non-limiting preferably biocompatible materials, such as, without limitation, polypropylene, polyester, polysulfone, nylon, polyurethane, polystyrene, polyethylene (PE) (including high-density and low-density PE's), polyethylene terephthalate (PET), polybutylene terephthalate (PBT), polytrimethylene terephthalate, polyetheretherketone (PEEK), poly(methyl methacrylate) (PMMA), polyethylene terephthalate (PET), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, ethylene vinyl acetate copolymers (EVA), silicones, polyethylene naphthalate (PEN), polyimide (PI), polyetherimide (PEI), polyphenylene sulfide (PPS), polyphenylene oxide (PPO), poly paraphenylene terephthalamide, perfluoro(propyl vinyl ether) (PFA), ethylene vinyl alcohol, polyolefin, epoxy, poly(styrene-b-isobutylene-b-styrene), polycarbonates, ionomers, or the like including mixtures, combinations, and copolymers thereof; a metal, such stainless steel, a nickel-titanium alloy such as Nitinol, or a cobalt-chromium-nickel based alloy such as Elgiloy®, or the like; a bioabsorbable or biodegradable material, such as polyglycolic acid, lactic acid, poly(lactic-co-glycolic acid), caprolactone, polymers, polydioxanone, cat or bovine intestine, or the like; a natural fiber, such as silk or cotton, or the like; or mixtures, composites, combinations, copolymers, or co-constructions of any of the above. Alternatively, the strut members may be formed by cutting (e.g., by laser-cutting) a tubular structure (e.g., an, optionally monolithic, cylindrical tubular member) into an expandable configuration, the cuts forming the strut members. Such tubular member may be formed from any suitable material of the above-listed materials as may be appreciated by one of ordinary skill in the art.

Instead of an expandable member, a tissue-engagement member of a locator system formed in accordance with various principles of the present disclosure may engage tissue at the target site by grasping the tissue. For instance, in the example of an embodiment of a locator system 400 illustrated in FIG. 9 and FIG. 10, a beacon 410 is delivered proximal to a tissue-engagement member 420 so that grasper arms 422 of the tissue-engagement member 420 extend distally to the distal end 401 of the locator system 400 to be engaged with tissue at the target site TS. As illustrated in FIG. 9 and FIG. 10, the beacon 410 is mounted on a flexible elongate member 430 (e.g., a braided hollow shaft) which may also deliver the beacon 410 to the target site TS (e.g., through a working channel or lumen within the delivery system 1000). A controller 440 may be operatively engaged with the grasper arms 422 to shift the grasper arms 422 between a closed configuration for delivery (as illustrated in FIG. 9), and an open configuration (as illustrated in FIG. 10) in which the grasper arms 422 may engage tissue at the target site TS therebetween along the distal ends 421 thereof. In some embodiments, the grasper arms have one or more additional grasping features, such as a sawtooth or crenulated profile 429, or teeth, such as along the distal end 421 and/or along edges thereof. Once tissue at the target site TS is positioned between the grasper arms 422, the controller 440 may be actuated to shift the grasper arms 422 to a closed configuration in which the grasper arms 422 are brought together to grasp tissue therebetween. The delivery system 1000 may then be withdrawn from the target site TS, in accordance with various principles of the present disclosure, without disturbing the position in which the tissue-engagement member 420 anchors the beacon 410 for identification of the location thereof for performance of a further procedure. The controller 440 may be actuated to shift the grasper arms 422 back to the delivery configuration to withdraw the tissue-engagement member 420 from the target site TS, such as upon completion of a procedure performed with respect to the target site TS.

In the example of an embodiment of a tissue-engagement member 420 illustrated in FIG. 9 and FIG. 10, the grasper arms 422 are pivotably coupled above a pivot 425a. In some embodiments, proximal ends 423 of the grasper arms 422 are pivotably coupled about pivots 425b to distal ends 427 of actuator arms 424. The proximal ends 429 of the actuator arms 424 may be coupled together within the distal end 431 of the flexible elongate member 430 about a further pivot 425c or may be joined together such as along a unitary bend. A spring 442 may be positioned between the pivot 425a (about which the grasper arms 422 are coupled) and the proximal end 403 of the tissue-engagement member 420 to hold the tissue-engagement member 420 in the closed configuration illustrated in FIG. 9. Proximal retraction of the actuator 440 draws the pivot 425a (about which the grasper arms 422 are coupled) closer to the proximal end 403 of the tissue-engagement member 420 to pivot the actuator arms 424 to cause the grasper arms 422 to shift into the open configuration illustrated in FIG. 10. In such embodiment, the spring 442 may be configured to bias the tissue-engagement member 420 into a closed configuration such that release of proximally directed force on the actuator 440 returns the tissue-engagement member 420 to the closed configuration. Alternatively or additionally, the proximal ends 429 of the actuator arms 424 may be coupled together along a unitary bend forming a spring biasing the actuator arms 424 apart when extended distally from within the distal end 431 of the sheath 430. The distal end 431 of the sheath 430 holds the actuator arms 424 in a closed configuration, and the closed configuration of the actuator arms 424 holds the grasper arms 422 in a closed configuration, as illustrated in FIG. 9. Distal advancement of the actuator 440 advances the proximal ends 429 of the actuator arms 424 out of the distal end 431 of the sheath 430 to allow the grasper arms 422 to move into the open configuration illustrated in FIG. 10. In such embodiment, the spring 442 may bias the grasper arms 422 into an open configuration as well, or need not be present, and the grasper arms 422 are closed by being withdrawn back into the sheath 430. It will be appreciated that other configurations of grasper arms 422 and controllers 440 are within the scope and spirit of the present disclosure, the present disclosure not being limited to the configuration of the example of an embodiment illustrated in FIG. 9 and FIG. 10.

In view of the above, in accordance with various principles of the present disclosure, as described above, a locator system is delivered to a target site and deployed in a manner which allows withdrawal of the delivery system without disturbing the locator system, i.e., without affecting the position at which the locator system has been deployed at the target site. A procedure may then be performed with respect to the target site with tools at another anatomical location in the body, with the aid of a visualization device capable of identifying the target site by locating the beacon of the locator system.

Although embodiments of the present disclosure may be described with specific reference to medical devices and systems (e.g., endoscopic devices, accessory tools, and/or guidewires) used in the GI system, it should be appreciated that locator devices and systems as described herein may be used with a variety of medical procedures performed in ductal, luminal, vascular, or body lumen anatomies, including, for example, interventional radiology procedures, balloon angioplasty/angiography procedures, thrombolysis procedures, urological or gynecological procedures, and the like. The medical devices herein may include a variety of medical devices for navigating body lumens, including, for example, catheters, ureteroscopes, bronchoscopes, colonoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

It will be appreciated that the foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features.

One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A locator system comprising:
a flexible elongate member;
a beacon mounted on said flexible elongate member; and
a tissue engagement member;
wherein said tissue engagement member is configured to engage tissue at a target site to anchor said locator system with respect to the target site.

2. The locator system of claim 1, wherein said tissue engagement member is expandable to engage tissue at the target site to anchor said locator system with respect to the target site.

3. The locator system of claim 2, wherein said tissue engagement member is an inflatable balloon.

4. The locator system of claim 2, wherein said tissue engagement member is an expandable stent.

5. The locator system of claim 2, wherein said tissue engagement member is mounted on said flexible elongate member.

6. The locator system of claim 1, wherein said tissue engagement member has a pair of grasper arms configured to grasp tissue at the target site therebetween.

7. The locator system of claim 1, further comprising a controller extending along said flexible elongate member and actuatable to shift said tissue engagement member between a delivery configuration and a tissue-engaging configuration.

8. The locator system of claim 7, wherein said tissue engagement member is inflatable, and said controller is an inflation lumen through said flexible elongate member.

9. The locator system of claim 7, wherein said tissue engagement member is inflatable, and said controller is an inflation lumen within an inflation line extending along said flexible elongate member.

10. The locator system of claim 7, wherein:
said controller is a sheath extending about said flexible elongate member; and
said tissue engagement member is an expandable stent positioned within said controller in the delivery configuration and expandable into the tissue-engaging configuration when outside said controller.

11. The locator system of claim 7, wherein said tissue engagement member has a pair of grasper arms configured to grasp tissue at the target site therebetween.

12. A locator system and delivery system therefor, comprising:
a beacon;
a tissue engagement member; and
a controller actuatable to shift said tissue engagement member between a delivery configuration in which said tissue engagement member is compact and deliverable transluminally to a target site, and a tissue-engaging configuration in which said tissue engagement member engages tissue at the target site to anchor said beacon with respect to the target site.

13. The locator system and delivery system therefor of claim 12, further comprising a flexible elongate member on which said beacon and said tissue engagement member are mounted.

14. The locator system and delivery system therefor of claim 12, wherein said tissue engagement member is an inflatable balloon, and said controller is an inflation lumen in fluid communication with said balloon.

15. The locator system and delivery system therefor of claim 12, wherein:
said controller is a sheath; and
said tissue engagement member is an expandable stent shiftable between a delivery configuration when within said sheath and an expanded tissue-engaging configuration when outside said sheath.

16. The locator system and delivery system therefor of claim 12, wherein said tissue engagement member comprises a pair of jaws movable by said controller between a closed configuration in which said jaws are in a delivery configuration or grasping tissue therebetween, and an open configuration in which said jaws may engage tissue therebetween.

17. A method of locating a target site in a first anatomical structure from within a second anatomical structure, said method comprising:
delivering a locator system to the target site with a delivery system;
anchoring the locator system with respect to the target site by engaging a tissue engagement member of the locator system with tissue at the target site;
withdrawing the delivery system from the locator system to leave the locator system in place anchored with respect to the target site; and
locating and identifying the locator system from the second anatomical structure, through a wall of at least the second anatomical structure.

18. The method of claim 17, further comprising forming an anastomosis between the first anatomical structure and the second anatomical structure along the target site.

19. The method of claim 17, further comprising expanding the tissue engagement member from a compact delivery configuration to an expanded configuration to engage tissue at the target site to anchor the locator system with respect to the target site.

20. The method of claim 17, further comprising anchoring the locator system to the target site by grasping tissue at the target site between grasper arms of the tissue engagement member.
